SEQUENCE LISTING (8 sequences)

SEQ ID NO: 1 - protein, 358 aa, Mus musculus
MAAAAAAGPEMVRGQVFDVGPRYTNLSYIGEGAYGMVCSAYDNLNKVRVAIKKISPFEHQTYCQRTLREIKILLRFRHENIIGINDIIRAPTIEQMKDVYIVQDLMETDLYKLLKTQHLSNDHICYFLYQILRGLKYIHSANVLHRDLKPSNLLLNTTCDLKICDFGLARVADPDHDHTGFLTEYVATRWYRAPEIMLNSKGYTKSIDIWSVGCILAEMLSNRPIFPGKHYLDQLNHILGILGSPSQEDLNCIINLKARNYLLSLPHKNKVPWNRLFPNADSKALDLLDKMLTFNPHKRIEVEQALAHPYLEQYYDPSDEPIAEAPFKFDMELDDLPKEKLKELIFEETARFQPGYRS

SEQ ID NO: 2 - protein, 379 aa, Homo Sapiens
MAAAAAQGGGGGEPRRTEGVGPGVPGEVEMVKGQPFDVGPRYTQLQYIGEGAYGMVSSAYDHVRKTRVAIKKISPFEHQTYCQRTLREIQILLRFRHENVIGIRDILRASTLEAMRDVYIVQDLMETDLYKLLKSQQLSNDHICYFLYQILRGLKYIHSANVLHRDLKPSNLLINTTCDLKICDFGLARIADPEHDHTGFLTEYVATRWYRAPEIMLNSKGYTKSIDIWSVGCILAEMLSNRPIFPGKHYLDQLNHILGILGSPSQEDLNCIINMKARNYLQSLPSKTKVAWAKLFPKSDSKALDLLDRMLTFNPNKRITVEEALAHPYLEQYYDPTDEPVAEEPFTFAMELDDLPKERLKELIFQETARFQPGVLEAP

SEQ ID NO: 3 - protein, 1563 aa, Homo Sapiens
MGSQALQEWGQREPGRWPDPAGKKDVRREASDSGRAGTWPRGPSECSPREKMAAAAGNRASSSGFPGARATSPEAGGGGGALKASSAPAAAAGLLREAGSGGRERADWRRRQLRKVRSVELDQLPEQPLFLAASPPASSTSPSPEPADAAGSGTGFQPVAVPPPHGAASRGGAHLTESVAAPDSGASSPAAAEPGEKRAPAAEPSPAAAPAGREMENKETLKGLHKMDDRPEERMIREKLKATCMPAWKHEWLERRNRRGPVVVKPIPVKGDGSEMNHLAAESPGEVQASAASPASKGRRSPSPGNSPSGRTVKSESPGVRRKRVSPVPFQSGRITPPRRAPSPDGFSPYSPEETNRRVNKVMRARLYLLQQIGPNSFLIGGDSPDNKYRVFIGPQNCSCARGTFCIHLLFVMLRVFQLEPSDPMLWRKTLKNFEVESLFQKYHSRRSSRIKAPSRNTIQKFVSRMSNSHTLSSSSTSTSSSENSIKDEEEQMCPICLLGMLDEESLTVCEDGCRNKLHHHCMSIWAEECRRNREPLICPLCRSKWRSHDFYSHELSSPVDSPSSLRAAQQQTVQQQPLAGSRRNQESNFNLTHYGTQQIPPAYKDLAEPWIQVFGMELVGCLFSRNWNVREMALRRLSHDVSGALLLANGESTGNSGGSSGSSPSGGATSGSSQTSISGDVVEACCSVLSMVCADPVYKVYVAALKTLRAMLVYTPCHSLAERIKLQRLLQPVVDTILVKCADANSRTSQLSISTLLELCKGQAGELAVGREILKAGSIGIGGVDYVLNCILGNQTESNNWQELLGRLCLIDRLLLEFPAEFYPHIVSTDVSQAEPVEIRYKKLLSLLTFALQSIDNSHSMVGKLSRRIYLSSARMVTTVPHVFSKLLEMLSVSSSTHFTRMRRRLMAIADEVEIAEAIQLGVEDTLDGQQDSFLQASVPNNYLETTENSSPECTVHLEKTGKGLCATKLSASSEDISERLASISVGPSSSTTTTTTTTEQPKPMVQTKGRPHSQCLNSSPLSHHSQLMFPALSTPSSSTPSVPAGTATDVSKHRLQGFIPCRIPSASPQTQRKFSLQFHRNCPENKDSDKLSPVFTQSRPLPSSNIHRPKPSRPTPGNTSKQGDPSKNSMTLDLNSSSKCDDSFGCSSNSSNAVIPSDETVFTPVEEKCRLDVNTELNSSIEDLLEASMPSSDTTVTFKSEVAVLSPEKAENDDTYKDDVNHNQKCKEKMEAEEEEALAIAMAMSASQDALPIVPQLQVENGEDIIIIQQDTPETLPGHTKAKQPYREDTEWLKGQQIGLGAFSSCYQAQDVGTGTLMAVKQVTYVRNTSSEQEEVVEALREEIRMMSHLNHPNIIRMLGATCEKSNYNLFIEWMAGGSVAHLLSKYGAFKESVVINYTEQLLRGLSYLHENQIIHRDVKGANLLIDSTGQRLRIADFGAAARLASKGTGAGEFQGQLLGTIAFMAPEVLRGQQYGRSCDVWSVGCAIIEMACAKPPWNAEKHSNHLALIFKIASATTAPSIPSHLSPGLRDVALRCLELQPQDRPPSRELLKHPVFRTTW

SEQ ID NO: 4 - protein, 720 aa, Mus musculus
MAEKFESLMNIHGFDLGSRYMDLKPLGCGGNGLVFSAVDNDCDKRVAIKKIVLTDPQSVKHALREIKIIRRLDHDNIVKVFEILGPSGSQLTDDVGSLTELNSVYIVQEYMETDLANVLEQGPLLEEHARLFMYQLLRGLKYIHSANVLHRDLKPANLFINTEDLVLKIGDFGLARIMDPHYSHKGHLSEGLVTKWYRSPRLLLSPNNYTKAIDMWAAGCIFAEMLTGKTLFAGAHELEQMQLILDSIPVVHEEDRQELLSVIPVYIRNDMTEPHRPLTQLLPGISREALDFLEQILTFSPMDRLTAEEALSHPYMSIYSFPTDEPISSHPFHIEDEVDDILLMDETHSHIYNWERYHDCQFSEHDWPIHNNFDIDEVQLDPRALSDVTDEEEVQVDPRKYLDGDREKYLEDPAFDTSYSAEPCWQYPDHHENKYCDLECSHTCNYKTRSSPYLDNLVWRESEVNHYYEPKLIIDLSNWKEQSKEKSDKRGKSKCERNGLVKAQIALEEASQQLAERERGQGFDFDSFIAGTIQLSAQHQSADVVDKLNDLNSSVSQLELKSLISKSVSREKQEKGRANLAQLGALYQSSWDSQFVSGGEECFLISQFCCEVRKDEHAEKENTYTSYLDKFFSRKEDSEMLETEPVEEGKRGERGREAGLLSGGGEFLLSKQLESIGTPQFHSPVGSPLKSIQATLTPSAMKSSPQIPHKTYSSILKHLN

SEQ ID NO: 5 - protein, 384 aa, Mus musculus
MSRSKRDNNFYSVEIGDSTFTVLKRYQNLKPIGSGAQGIVCAAYDAILERNVAIKKLSRPFQNQTHAKRAYRELVLMKCVNHKNIIGLLNVFTPQKSLEEFQDVYIVMELMDANLCQVIQMELDHERMSYLLYQMLCGIKHLHSAGIIHRDLKPSNIVVKSDCTLKILDFGLARTAGTSFMMTPYVVTRYYRAPEVILGMGYKENVDLWSVGCIMGEMVCHKILFPGRDYIDQWNKVIEQLGTPCPEFMKKLQPTVRTYVENRPKYAGYSFEKLFPDVLFPADSEHNKLKASQARDLLSKMLVIDASKRISVDEALQHPYINVWYDPSEAEAPPPKIPDKQLDEREHTIEEWKELIYKEVMDLEERTKNGVIRGQPSPLAQVQQ

SEQ ID NO: 6 - protein, 367 aa, Homo Sapiens
MSSPPPARSGFYRQEVTKTAWEVRAVYRDLQPVGSGAYGAVCSAVDGRTGAKVAIKKLYRPFQSELFAKRAYRELRLLKHMRHENVIGLLDVFTPDETLDDFTDFYLVMPFMGTDLGKLMKHEKLGEDRIQFLVYQMLKGLRYIHAAGIIHRDLKPGNLAVNEDCELKILDFGLARQADSEMTGYVVTRWYRAPEVILNWMRYTQTVDIWSVGCIMAEMITGKTLFKGSDHLDQLKEIMKVTGTPPAEFVQRLQSDEAKNYMKGLPELEKKDFASILTNASPLAVNLLEKMLVLDAEQRVTAGEALAHPYFESLHDTEDEPQVQKYDDSFDDVDRTLDEWKRVTYKEVLSFKPPRQLGARVSKETPL

SEQ ID NO: 7 - protein, 361 aa, Danio Rerio
MSQKARPTFYRQELNKTIWEVPERYQNLSPVGSGAYGSVCSAFDSKAGLRVAVKKLSRPFQSIIHAKRTYRELRLLKHMKHENVIGLLDVFSPATSLEEFNDVYLVTHLMGADLNNIVKCQKLTDDHVQFLIYQILRALKYIHSADIIHRDLKPSNLAVNEDCELKILDFGLARLTDDEMTGYVATRWYRAPEIMLNWMHYNMTVDIWSVGCIMAELLTGRTLVSRTDHIDQLKLIMMLVGTPGPELLMKISSESARNYISSLPHMPKRNFADVFIGANPLAVDLLEKMLVLDTDKRITASQALAHPYFAQYHDPDDEPEADPYDQSFESRDLEIEEWKSLTYEEVVSFEPPVFDGDEMES

SEQ ID NO: 8 - protein, 307 aa, Homo Sapiens
MSQERPTFYRQELNKTIWEVPERYQNLSPVGSGAYGSVCAAFDTKTGLRVAVKKLSRPFQSIIHAKRTYRELRLLKHMKHENVIGLLDVFTPARSLEEFNDVYLVTHLMGADLNNIVKCQKLTDDHVQFLIYQILRGLKYIHSADIIHRDLKPSNLAVNEDCELKILDFGLARHTDDEMTGYVATRWYRAPEIMLNWMHYNQTVDIWSVGCIMAELLTGRTLFPGTDHIDQLKLILRLVGTPGAELLKKISSESLSTCWRRCLYWTQIRELQRPKPLHMPTLLSTTILMMNQWPILMISPLKAGTSL

(19) United States
(12) Patent Application Publication
(10) Pub. No.: US 2005/0095657 A1
(43) Pub. Date: May 5, 2005

(54) Methods and kits for detecting proteins

(76) Inventors: Jack L. Arbiser, Atlanta, GA (US); Cynthia Cohen, Atlanta, GA (US)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 10/945,684

(22) Filed: Sep. 21, 2004

Related U.S. Application Data
(63) Continuation of application No. PCT/US03/32248, filed on Oct. 14, 2003.
(60) Provisional application No. 60/418,038, filed on Oct. 11, 2002.

(51) Int. Cl. G01N 33/53; G01N 33/537; G01N 33/543

(52) U.S. Cl. 435/7.92

(57) ABSTRACT

Briefly described, methods of detecting a phosphorylated mitogen activated protein kinase (P-MAPK), methods of diagnosing cancer, kits for detecting P-MAPK, and kits for diagnosing cancer, are disclosed. One exemplary kit, among others, includes a composition including an antibody that bonds to a phosphorylated mitogen activated protein kinase (P-MAPK) or a variant thereof to form a detectable complex; and a set of printed instructions specifying, in order of implementation, steps to be followed for detecting the P-MAPK or a variant thereof by detecting the complex. The composition and the printed instructions are in packaged combination.

### CROSS-REFERENCE TO RELATED APPLICATION

This application claims priority to copending PCT Application entitled “Methods and Kits for Detecting Proteins”, having PCT No. US03/32248, filed Oct. 14, 2003 and U.S. provisional application entitled, “Isolation of Angiogenesis Inhibitors From Mate Tea”, having Ser. No. 60/418,038, filed Oct. 11, 2002, which is entirely incorporated herein by reference.

### STATEMENT REGARDING FEDERALLY SPONSORED RESEARCH OR DEVELOPMENT

Aspects of the work described herein may have been supported by National Institutes of Health, grant number R01AR47901. Therefore, the U.S. government may have certain rights in the invention.

### TECHNICAL FIELD

The present invention relates generally to detection of proteins, and more particularly to detecting proteins related to cancer and diagnosing cancer.

### BACKGROUND

Cancer can be defined as an abnormal growth of tissue characterized by a loss of cellular differentiation. This term encompasses a large group of diseases in which there is an invasive spread of undifferentiated cells from a primary site to other parts of the body where further undifferentiated cellular replication occurs, which eventually interferes with the normal functioning of tissues and organs.

Cancer can be defined by four characteristics which differentiate neoplastic cells from normal ones: (1) clonality-cancer starts from genetic changes in a single cell which multiplies to form a clone of neoplastic cells; (2) autonomy-biochemical and physical factors that normally regulate cell growth, do not do so in the case of neoplastic cells; (3) anaplasia-neoplastic cells lack normal differentiation which occurs in nonmalignant cells of that tissue type; (4) metastasis-neoplastic cells grow in an unregulated fashion and spread to other parts of the body.

Each cancer is characterized by the site, nature, and clinical cause of undifferentiated cellular proliferation. The underlying mechanism for the initiation of cancer is not completely understood; however, about 80% of cancers may be triggered by external stimuli such as exposure to certain chemicals, tobacco smoke, ultra violet rays, ionizing radiation, and viruses. Development of cancer in immunosuppressed individuals indicates that the immune system is an important factor controlling the replication and spread of cancerous cells throughout the body.

The high incidence of cancer in certain families, though, suggests a genetic disposition towards development of cancer. The molecular mechanisms involved in such genetic dispositions fall into a number of classes including those that involve oncogenes and suppressor genes.

Proto-oncogenes are genes that code for growth promoting factors necessary for normal cellular replication. Due to mutation, such proto-oncogenes are inappropriately expressed and are then termed oncogenes. Oncogenes can be involved in malignant transformation of the cell by stimulating uncontrolled multiplication.

Suppressor genes normally act by controlling cellular proliferation through a number of mechanisms including binding transcription factors important to this process. Mutations or deletions in such genes contribute to malignant transformation of a cell.

Malignant transformation develops and cancer results because cells of a single lineage accumulate defects in certain genes such as proto-oncogenes and suppressor genes responsible for regulating cellular proliferation. A number of such specific mutations and/or deletions must occur in a given cell for initiation of uncontrolled replication. It is believed that genetic predisposition to a certain type of cancer results from inheritance of genes that already have a number of mutations in such key regulatory genes and subsequent exposure to environmental carcinogens causes enough additional key mutations or deletions in these genes in a given cell to result in malignant transformation. Changes in other types of genes could further the ability of tumors to grow, invade local tissue, and establish metastases at distant body sites.

Melanoma is a classic example of tumor progression. At least some cutaneous melanomas are thought to arise from precursor lesions termed atypical nevi. Patients with germ-line mutations in the tumor suppressor gene p16^{ink4a} have an increased rate of melanoma, suggesting that loss of this tumor suppressor gene is involved in melanoma progression; however, the point at which p16^{ink4a} is lost is not clear. Clinically the transition from atypical nevus to radial growth melanoma has been observed, as has the transition from radial growth melanoma to vertical growth melanoma. Various mutations have been observed in late-stage melanoma, such as activation of ras or loss of the tumor suppressor gene PTEN. However, the alterations in signal transduction, which accompany the transition from atypical nevus to radial growth melanoma are not well understood.

It should also be noted that the major cause of malpractice lawsuits for anitomical pathology is misdiagnosis of melanoma. Therefore, there is a need in the industry for a method of diagnosing cancers and, in particular, diagnosing melanoma.

### SUMMARY

Briefly described, embodiments of this disclosure, among others, include methods of detecting a phosphorylated mitogen activated protein kinase (P-MAPK), methods of diagnosing cancer, kits for detecting P-MAPK, and kits for diagnosing cancer. One exemplary kit, among others, includes a composition including an antibody that bonds to a phosphorylated mitogen activated protein kinase (P-MAPK) or a variant thereof to form a detectable complex; and a set of printed instructions specifying, in order of implementation, steps to be followed for detecting the P-MAPK or a variant thereof by detecting the complex. The composition and the printed instructions are in packaged combination.

Methods of detecting P-MAPK are also provided. One exemplary method includes, among others: providing a sample; contacting the sample with at least one antibody having an affinity for the phosphorylated portion of P-MAPK; forming an antibody/P-MAPK complex; and detecting the antibody/P-MAPK complex, wherein the presence of the antibody/P-MAPK complex indicates that the P-MAPK is present in the sample, and wherein constitutive expression of P-MAPK is indicative of cancer.

In addition, methods of diagnosing cancer are provided. One exemplary method includes, among others: providing a sample from a subject; and determining the presence of a phosphorylated mitogen activated protein kinase (P-MAPK) or a variant thereof in the sample, and wherein constitutive expression of P-MAPK in the sample is indicative of cancer.

### BRIEF DESCRIPTION OF THE FIGURES

Many aspects of the invention can be better understood with reference to the following drawings. The components in the drawings are not necessarily to scale.

FIG. 1 illustrates an immunohistochemical and histologic analysis of nevi and melanoma. The top row (A-D) represents immunohistochemistry for P-MAPK (A), VEGF (B), CD31 (C) and TF (D) in an atypical nevus. The second row represents immunohistochemistry for P-MAPK (E), VEGF (F), CD31 (G), and TF (H) in a radial growth melanoma. The third row represents immunohistochemistry for P-MAPK (I), VEGF (J), CD31 (K), and TF (L) in a vertical growth melanoma.

FIG. 2 illustrates a western blot analysis of P-MAPK and MAPK expression in primary melanocytes (Lane 1) and PMWK radial growth melanoma cells (Lane 2). Equal amounts of protein from primary melanocytes cultured for 24 hours in DMEM supplemented with 5% fetal calf serum were analyzed using antibodies specific for phosphorylated p42/44 MAP kinase (P-MAPK) and total MAP kinase (MAPK).

### DETAILED DESCRIPTION

Briefly described, embodiments of this disclosure provide diagnostic methods and diagnostic kits that can be used to determine if a sample includes cancerous cells. In addition, embodiments of this disclosure provide methods and kits for the detection of a phosphorylated mitogen activated protein kinase (P-MAPK), wherein constitutive expression of P-MAPK is indicative of cancer. Furthermore, embodiments of this disclosure provide methods and kits for the detection of the P-MAPK and a second protein selected from VEGF and TF, wherein constitutive expression of P-MAPK and expression of the activated second protein is indicative of cancer.

Prior to describing the various embodiments in additional detail, the following definitions are provided to facilitate the description of the embodiments.

Definitions

As used herein, the following terms have the given meanings unless expressly stated to the contrary.

The term “polypeptides” includes proteins and fragments thereof and antibodies and fragments thereof Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

“Variant” refers to a polypeptide that differs from a reference polypeptide, but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

Modifications and changes can be made in the structure of the polypeptides of in disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly, where the biological functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamnine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of this disclosure thus contemplate functional or biological equivalents of a polypeptide as set forth above. In particular, embodiments of the polypeptides can include variants having about 50%, 60%, 70%, 80%, 90%, and 95% sequence identity to the polypeptide of interest.

“Identity,” as known in the art, is a relationship between two or more polypeptide sequences, as determined by comparing the sequences. In the art, “identity” also means the degree of sequence relatedness between polypeptide as determined by the match between strings of such sequences. “Identity” and “similarity” can be readily calculated by known methods, including, but not limited to, those described in (Computational Molecular Biology, Lesk, A. M, Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M, and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J Applied Math., 48:1073 (1988).

Preferred methods to determine identity are designed to give the largest match between the sequences tested. Methods to determine identity and similarity are codified in publicly available computer programs. The percent identity between two sequences can be determined by using analysis software (i.e., Sequence Analysis Software Package of the Genetics Computer Group, Madison Wis.) that incorporates the Needelman and Wunsch, (J. Mol. Biol., 48: 443-453, 1970) algorithm (e.g., NBLAST, and XBLAST). The default parameters are used to determine the identity for the polypeptides of the present invention.

By way of example, a polypeptide sequence may be identical to the reference sequence, that is be 100% identical, or it may include up to a certain integer number of amino acid alterations as compared to the reference sequence such that the % identity is less than 100%. Such alterations are selected from: at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the reference polypeptide by the numerical percent of the respective percent identity (divided by 100) and then subtracting that product from said total number of amino acids in the reference polypeptide.

The term “antibody” is used in the broadest sense and specifically covers monoclonal antibodies, polyclonal antibodies, multispecific antibodies (e.g., bispecific antibodies), and antibody fragments so long as they bind specifically to a target antigen.

The term “primary antibody” herein refers to an antibody that has an affinity (e.g., binds specifically) for the phosphorylated portion of the target protein (e.g., phosphorylated mitogen activated protein kinase) and to the substantial exclusion of other proteins in a sample. The primary antibody can be bound to a label that can be used to detect the primary antibody.

The term “secondary antibody” herein refers to an antibody that binds specifically to a primary antibody, thereby forming a bridge between the primary antibody and the target protein. The secondary antibody can be bound to a label that can be used to detect the secondary antibody.

The word “label” when used herein refers to a reagent, compound, composition, complex, or particle, which is bound (e.g., conjugated or fused directly or indirectly to the antibody) to an antibody and facilitates detection of the antibody to which it is bound. The label may itself be detectable (e.g., radioisotope labels or fluorescent labels) or, in the case of an enzymatic label, may catalyze chemical alteration of a substrate compound or composition that is detectable.

Discussion

In general, embodiments of the methods described herein provide processes of screening biological samples for the presence of phosphorylated mitogen activated protein kinase (P-MAPK) corresponding to MAPK and variants thereof. In addition, embodiments of the methods described herein provide processes of screening biological samples for the presence of P-MAPK corresponding to MAPK and variants thereof and a second protein selected from vascular endothelial growth factor (VEGF) and tissue factor (TF), wherein constitutive expression of P-MAPK and expression of the second protein are indicative of cancer.

MAPK is a regulatory protein in a signal transduction pathway operative in cancers such as melanoma, for example. Antibodies specific to P-MAPK were studied in a series of melanomas and atypical nevi (non-malignant precursors to melanoma). Expression of activated MAPK and its targets, VEGF and TF, are observed in radial growth melanoma and later stages, but not in its immediate precursors. Constitutive expression of activated MAPK is observed in radial growth melanoma cells compared with primary melanocytes. This indicates that MAPK activation is an early event in melanoma progression. Therefore, detection of P-MAPK and variants thereof (and/or VEGF and/or TF and variats of each) in a sample indicates the sample is cancerous. Moreover, detection of P-MAPK (and/or VEGF and/or TF and variats of each) can be used as a diagnostic test for screening samples for the presence of cancer because constitutive expression of P-MAPK (and/or and expression of the activated second protein) is indicative of cancer. Additional details are described in Example 1 below.

The MAPK family includes regulatory proteins that are known to regulate cellular responses to both proliferative and stress signals. MAPK is abundantly expressed in nerve cells. There are three distinct groups of MAPKs in mammalian cells: a) extracellular signal-regulated kinases (ERKs), b) c-Jun N-terminal kinases (JNKs) and c) stress activated protein kinases (SAPKs). MAPKs include, but are not limited to, MAPK-1 (SEQ ID NO:1, Protein ID: NP 036079.1, corresponding coding sequence Accession No. NM 011949), MAPK-3 (SEQ ID NO:2, Protein ID: XP 055766.3, corresponding coding sequence Accession No. XM 055766), MAP3K1 (SEQ ID NO:3, Protein ID: XP 042066.7, corresponding coding sequence Accession No. XM 042066), MAPK-6 (SEQ ID NO:4, Protein ID: NP 056621.2, corresponding coding sequence Accession No. NM 015806), MAPK-8 (SEQ ID NO:5, Protein ID: NP 057909.1, corresponding coding sequence Accession No. NM 016700), MAPK-12 (SEQ ID NO:6, Protein ID: AAH15741.1 055766.3, corresponding coding sequence Accession No. BC 015741), MAPK-14 (SEQ ID NO:7, Protein ID: AA 091248.1, corresponding coding sequence Accession No. AY 391436), MAPK-14 (transcript variant 4) (SEQ ID NO:8, Protein ID: NP 620583.1, corresponding coding sequence Accession No. NM 139014), each proteins homologues and isoforms, and each proteins variants.

As indicated above, P-MAPK's include phosphorylated MAPK's and variants thereof P-MAPK's are defined as MAPK's having one or more phosphorylated amino acids (e.g., serine, threonine, and tyrosine). The P-MAPK's correspond to phosphorylated MAPK-1 (SEQ ID NO:1), phosphorylated MAPK-3 (SEQ ID NO:2), phosphorylated MAP3K1 (SEQ ID NO:3), phosphorylated MAPK-6 (SEQ ID NO:4), phosphorylated MAPK-8 (SEQ ID NO:5), phosphorylated MAPK-12 (SEQ ID NO:6), phosphorylated MAPK-14 (SEQ ID NO:7), and phosphorylated MAPK-14 (SEQ ID NO:8).

The biological sample can be collected from a mammal such as, but not limited to, rats, mice, hamsters, rabbits, cats, dogs, pigs, sheep, cows, horses, primates, and humans. The sample can be a biological fluid (e.g., extracellular or intracellular fluid) or a cell extract, a tissue extract, or a homogenate. A biological sample can also be an isolated cell (e.g., in culture) or a collection of cells such as in a tissue sample or histology sample. A tissue sample can be suspended in a liquid medium or fixed onto a solid support.

In general, P-MAPK (e.g., corresponding to MAPK and variants thereof) can be detected by exposing the sample to a primary antibody solution having one or more primary antibodies disposed therein. The primary antibodies have an affinity for the phosphorylated portion of the P-MAPK and conjugates with P-MAPK after incubation with the sample. In addition, a second protein selected from VEGF and TF and variants of each can be detected by exposing the sample to antibodies having an affinity for VEGF and TF by following similar methods and using similar kits as described herein for P-MAPK. For clarity, additional details in regard to VEGF and TF will not be discussed in additional detail.

The primary antibodies can include antibodies corresponding to P-MAPK described above. In particular, the primary antibodies can include antibodies such as, but not limited to, Phospho-Akt (Ser473) (4E2) Monoclonal Antibody (Biotinylated), Phospho-Akt (Ser473) Antibody, Phospho-Akt (Ser473) Antibody (IHC Specific), Phospho-Akt (Thr308) Antibody, Phospho-Akt Pathway Sampler Kit, Phospho-Akt (Ser473) (587F11) Monoclonal Antibody, Phospho-beta Catenin (Ser33/37/Thr41) Antibody, Phospho-beta Catenin (Thr41/Ser45) Antibody, Phospho-EGF Receptor (Tyr1045) Antibody, Phospho-EGF Receptor (Tyr1068) (1H12) Monoclonal Antibody, Phospho-EGF Receptor (Tyr1068) Antibody, Phospho-EGF Receptor (Tyr845) Antibody, Phospho-EGF Receptor (Tyr992) Antibody, Phospho-Elk-1 (Ser383) (2B1) Monoclonal Antibody, Phospho-FGF Receptor (Tyr653/654) Antibody, Phospho-FKHR (Thr24)/FKHRL1 (Thr32) Antibody, Phospho-GSK-3alpha (Ser21) (46H12) Monoclonal Antibody, Phospho-GSK-3alpha/beta (Ser21/9) Antibody, Phospho-GSK-3beta (Ser9) Antibody, Phospho-HER2/ErbB2 Antibody Sampler Kit, Phospho-IkappaB-alpha (Ser32) Antibody, Phospho-IkappaB-alpha (Ser32/36) (5A5) Monoclonal Antibody, Phospho-c-Jun (Ser63) II Antibody, Phospho-c-Jun (Ser73) Antibody, Phospho-p44/42 MAP Kinase (Thr202/Tyr204) Antibody Kit, Phospho-MEK1 (Ser298) Antibody, Phospho-MEK1 (Ser286) Antibody, Phospho-MEK1/2 (Ser217/221) Antibody, PhosphoPlus MEK1/2 (Ser217/221) Antibody Kit, PhosphoPlus MKK3/MKK6 (Ser189/207) Antibody Kit, PhosphoPlus MKK7 (Ser271/Thr275) Antibody, Immobilized Phospho-p38 MAPK (Thr180/tyr182 Monoclonal Antibody, Phospho-p38 MAP Kinase (Thr180/tyr182) (28B10) Monoclonal Antibody, Phospho-p53 (Ser15) Antibody, Phospho-p53 (Ser20) Antibody, Phospho-p70 S6 Kinase (Thr389) (1A5) Monoclonal Antibody, Phospho-p70 S6 Kinase (Thr389) Antibody, Phospho-p70 S6 Kinase (Thr421/Ser424) Antibody, Phospho-p70 S6 Kinase (Thr389, Thr421/Ser424) Antibody Kit, Phospho-p70 S6 Kinase Ser371) Antibody, Phospho (Tyr) p85 P13K Binding Montif Antibody, Phospho-PDGR Receptor beta (Tyr752) Antibody, Phospho-PDGR Receptor beta (Tyr752) (88H8) Monocional Antibody, Phospho-PDK1 (Ser241) Antibody, Phospho-PDK1 (Tyr373/376) Antibody, Phospho-PKCzeta/lambda (Thr410/403) Antibody, Phospho-PTEN (Ser380) Antibody, Phospho-Rac1/cdc42 (Ser71) Antibody, Phospho-Raf (Ser259) Antibody, Phospho-Raf (Ser338) Antibody, Phospho-SAPK/JNK (Thr183/Tyr185) (G9) Monoclonal Antibody, Phospho-SAPK/JNK (Thr183/Tyr185) (G9) Antibody, Phospho-SAPK/JNK Pathway Sampler Kit, Phospho-SEK1/MKK4 (Ser80) Antibody, Phospho-SEK1/MKK4 (Thr261) Antibody, Phospho-VEGF Receptor-2 (Tyr951) Antibody, Phospho-VEGF Receptor-2 (Tyr996) Antibody, and combinations thereof (All are available from Cell Signaling Technology Inc.).

Once the primary antibody solution is mixed with the sample and allowed to incubate, P-MAPK and the primary antibody form a primary antibody/P-MAPK complex. The primary antibody/P-MAPK complex can be detected in a manner described below. Detection of the primary antibody/P-MAPK complex indicates that the sample includes P-MAPK and that the sample contains cancerous cells because constitutive expression of P-MAPK is indicative of cancer.

Examples of cancers and cancer-related conditions detectable by embodiments of this disclosure include, but are not limited to, histologic types of cancer such as melanoma, carcinoma, sarcoma, mesothelioma, and lymphoma including precancerous lesions. These cancers can develop at one or more bodily sites and these include, but are not limited to, head and neck, oral cavity and pharynx (e.g., tongue, mouth, and pharynx), cancer of the digestive system (e.g., esophagus, stomach, small intestine, colon, rectum, anus, anal canal, anorectum, liver and intrahepatic bile ducts, gallbladder and other sites in the biliary tree, pancreas and other digestive organs), respiratory system (e.g., larynx, lungs, bronchi, and other respiratory organs), bones and joints, soft tissues (e.g., heart), skin (e.g., basal cell, squamous cell and melanoma), breast, genital system (e.g., prostate, testis, penis, and other male genital organs as well as uterine cervix, uterine corpus, ovary, vulva, vagina and other female genital organs), urinary system (e.g., urinary bladder, kidney and renal pelvis, urethra and other urinary organs), brain and nervous system, eye and orbit, and endocrine system (e.g., thyroid and other endocrine). In addition, cancer can include non-site specific cancers such as, but not limited to, lymphoma (e.g., Hodgkin's disease and non-Hodgkin's lymphoma), leukemia (e.g., acute lymphocytic leukemia, chronic lymphocytic leukemia, acute myeloid leukemia, chronic myeloid leukemia, and other forms of leukemia), and multiple melanoma. In particular, the cancers and cancer-related conditions detectable by embodiments of this disclosure include melanoma.

In some embodiments, the primary antibody and/or the P-MAPK can be immobilized (i.e., reversibly immobilized, irreversibly immobilized, or both) on a solid support. For example, the primary antibody and/or the P-MAPK can be immobilized to the solid support to perform different types of analysis such as, but not limited to, immunoassays (e.g., enzyme linked immuno sorbent assays (ELISAs)), western blot analysis, immunocyhtochemistry, immunohistochemistry, and immunochromatographic assays.

The solid support can include, but is not limited to, a plastic (e.g., polystyrene or cyclo-olefin polymers) a glass, a magnetic compound, a membrane (e.g., nylon or nitrocellulose) or other appropriate solid substrate for a particular application. A solid support can take a plurality of configuration, such as, but not limited to, beads, sheets, tubes, plates and/or wells (e.g., microtiter plates), columns, dipsticks, or other structures appropriate for a particular application.

In general, the primary antibody can be detected using systems such as, but not limited to, a fluorescence system, a chemiluminescence system, a phosphorescence system, an enzymatic reaction system, a colorimetry system, radiography system, a mass spectroscopy system, and a gel electrophoresis system. The primary antibody can be detected using direct and/or indirect detection techniques. Direct determination uses a primary antibody including a label (e.g., a fluorescent tag or an enzyme-label) that can be detected without further antibody interaction.

Numerous labels are available and include, but are not limited to, radioisotope labels (e.g., ³⁵S, ¹⁴C, ³H, and ¹³¹I), metal particle labels (e.g., colloidal gold particles, metal nanoparticles, and quantum dots), fluorescent labels (e.g., rare earth chelates (europium chelates), Texas Red, rhodamine, fluorescein, dansyl, Lissamine, umbelliferone, phycocrytherin, phycocyanin, or commercially available fluorophores), and enzyme-substrate labels (e.g., luciferin, 2,3-dihydrophthalazinediones, malate dehydrogenase, urease, and peroxidase (e.g., horseradish peroxidase (HRPO), alkaline phosphatase, beta-galactosidase, glucoamylase, lysozyme, saccharide oxidases (e.g., glucose oxidase, galactose oxidase, and glucose-6-phosphate dehydrogenase), heterocyclic oxidases (e.g., uricase and xanthine oxidase), lactoperoxidase, and microperoxidase)). Detection and/or quantitation techniques known to one skilled in the art (e.g., fluorimeter, spectrophotometer, and chemiluminometer) can be used to detect and/or quantitate the labels to identify the presence of the primary antibody.

For indirect determination the primary antibody can be conjugated with biotin, and any of the labels mentioned above can be conjugated with avidin, or vice versa. Biotin binds selectively to avidin and thus, the label can be conjugated with the primary antibody in this indirect manner. Alternatively, to achieve indirect conjugation of the label with the primary antibody, the primary antibody is conjugated with a small hapten and the label is conjugated with an anti-hapten antibody. Thus, indirect conjugation of the label with the primary and/or secondary antibody can be achieved.

In another embodiment, P-MAPK (e.g., corresponding to MAPK and variants thereof) can be detected by exposing the sample to a primary antibody solution having one or more primary antibodies disposed therein. The primary antibodies have an affinity for the phosphorylated portion of the P-MAPK and conjugates with P-MAPK upon mixture with the sample. Once the primary antibody solution is mixed with the sample and allowed to incubate, P-MAPK and the primary antibody form a primary antibody/P-MAPK complex. Next, the primary antibody/P-MAPK complex is exposed to a secondary antibody solution including one or more secondary antibodies having an affinity for the primary antibody. The secondary antibodies can include antibodies having an affinity for the primary antibodies described above.

Upon mixing the primary antibody/P-MAPK complex with the secondary antibody solution, a primary antibody/P-MAPK/secondary antibody complex is formed. The primary antibody/P-MAPK/secondary antibody complex can be detected in a manner described below. Detection of the primary antibody/P-MAPK/secondary antibody complex indicates that the sample includes P-MAPK and that the sample contains cancerous cells because constitutive expression of P-MAPK is indicative of cancer.

The primary antibody, the secondary antibody, and/or the P-MAPK can be immobilized (i.e., reversibly immobilized, irreversibly immobilized or both) on a solid support, as described above. However, both the primary antibody and the secondary antibody both can not be irreversibly immobilized on a solid support. The primary antibody and/or the secondary antibody can be immobilized to the solid support to perform different types of analysis such as, but not limited to, immunoassays (e.g., enzyme linked immuno sorbent assays (ELISAs)), western blots, immunocyhtochemistry, immunohistochemistry and immunochromatographic assays.

As indicated above, the primary antibody and/or the secondary antibody can be detected using direct and/or indirect detection techniques. In typical indirect detection techniques, the primary antibody (unlabeled) binds to P-MAPK and then a labeled secondary antibody binds-to the primary antibody. In another embodiment, the primary antibody and the secondary antibody are each labeled and therefore each antibody can be individually detected. In still another embodiment, the label can be indirectly attached to the primary antibody and/or secondary antibody. The primary antibody and the secondary antibody can use labels as described above.

For example, the primary and/or secondary antibody can be conjugated with biotin, and any of the labels mentioned above can be conjugated with avidin, or vice versa. Biotin binds selectively to avidin and thus, the label can be conjugated with the primary and/or secondary antibody in this indirect manner. Alteratively, to achieve indirect conjugation of the label with the primary and/or secondary antibody, the primary and/or secondary antibody is conjugated with a small hapten and the label is conjugated with an anti-hapten antibody. Thus, indirect conjugation of the label with the primary and/or secondary antibody can be achieved.

Another embodiment provides a kit for the detection or identification of P-MAPK (corresponding to MAPK and variants thereof). An exemplary kit contains a solution, for example in a buffer solution, the primary antibody that has an affinity for the phosphorylated portion of the P-MAPK. The kit also contains printed instructions for performing a protocol using the disclosed solution. In addition, the kit can optionally include components such as, but not limited to, reagents, buffers, developers, and other items known in the art to facilitate the detection of primary antibodies. The components included in the kit depend upon the type of analysis to be performed and the detection technique utilized.

In another embodiment, a kit is provided for the detection or identification of P-MAPK (corresponding to MAPK and variants thereof). An exemplary kit contains a solution including the primary antibody that has an affinity for the phosphorylated portion of the P-MAPK. In addition, the kit includes a solution including the secondary antibody that has an affinity for the primary antibody. The kit also contains printed instructions for performing a protocol using the disclosed solutions. Furthermore, the kit can optionally include components such as, but not limited to, reagents, buffers, developers, and other items known in the art to facilitate the detection of primary antibodies. The components included in the kit depend upon the type of analysis to be performed and the detection technique utilized.

In addition, since the identification of P-MAPK (corresponding to MAPK and variants thereof) in a sample is indicative that the sample includes cancerous cells, then the kit can also be used as a diagnostic kit to detect the presence of cancerous cells because constitutive expression of P-MAPK is indicative of cancer. The diagnostic kit includes one or more solutions having the primary antibody and/or a solution having the secondary antibody. The diagnostic kit also contains printed instructions for performing a protocol using the disclosed solutions. In addition, the kit can optionally include components such as, but not limited to, reagents, buffers, developers, and other items known in the art to facilitate the detection of primary antibodies. The components included in the kit depend upon the type of analysis to be performed and the detection technique utilized.

### EXAMPLE 1

The following is a non-limiting illustrative example of an embodiment of the present invention that is described in more detail in Cohen, et al., Clinical Cancer Research, 8, 3728 (2002), which is incorporated herein by reference. This example is not intended to limit the scope of any embodiment of this disclosure, but rather is intended to provide specific experimental conditions and results. Therefore, one skilled in the art would understand that many experimental conditions can be modified, but it is intended that these modifications be within the scope of the embodiments of this disclosure.

Materials and Methods

Formalin-fixed, paraffin-embedded blocks (117 malignant melanoma and 14 nevi) from the archives of the Department of Pathology and Laboratory Medicine at Emory University Hospital, Atlanta, Ga., were studied. Clinical, pathologic, and follow-up information was obtained from surgical pathology reports and the Winship Cancer Center Oncology Data Bank, Emory University School of Medicine, Atlanta, Ga. The nevi studied were composed of 3 junctional, 4 minimal atypical, 3 mildly atypical, and 3 moderate-severe atypia.

Immunohistochemistry: Five-μm sections were immunostained for P-MAPK (1/30; New England BioLabs, Beverly, Mass.), VEGF (1/160; Santa Cruz Biotechnologies, Santa Cruz, Calif.), TF (1/160; American Diagnostica Greenwich, Conn.), and CD31 (1/80; Dako Corporation, Santa Barbara, Calif.) using an avidin-biotin complex method, steam heat-induced epitope retrieval, and the DAKO Autostainer (Dako). An avidin-biotinylated enzyme complex kit (LSAB 2; Dako) was used according to the manufacturer's specifications with hematoxylin as counterstain. Positive controls were a hemangioma for P-MAPK, myometrial blood vessels (VEGF, CD31, and P-MAPK), and a known TF-positive breast carcinoma. Negative controls had the primary-specific antibody replaced by buffer. Specificity of the P-MAPK antiserum has been demonstrated previously using melanoma protein (Arbiser, et al., J. Am. Acad. Dermatol., 44, 1 (2001)). P-MAPK, VEGF, and TF were quantitated as intensity of immunostain (0-3+) and percentage of immunoreactive MM/nevi cells (0-100%). CD31 was visually semiquantitated as mean and maximum vessel density by two pathologists (A. Z-R and C. C.) in two “hot spots” at ×200 magnification, who viewed the slides at the same time but counted them independently, and the MVD was calculated as the average of the measurement of the pathologists.

Cell Culture/Western Blot Analysis: PMWK is a radial growth melanoma cell line characterized previously (Byers et al., Am. J. Pathol., 139, 423 (1991)). Primary human melanocytes were obtained from the Emory Skin Disease Research Center Tissue Culture Core and cultured in melanocyte growth medium until growth factor-deprivation experiments were performed. Western blot analysis of the active P-MAPK and total MAPK was performed on lysates of primary melanocytes and radial growth PMWK melanoma cells grown in the same medium (DMEM) supplemented with 5% FCS for 24 hours in the absence of exogenous growth factors. The specificity of the antibody has been demonstrated previously on melanoma lysates protein (Arbiser, et al., J. Am. Acad. Dermatol., 44, 1 (2001)). Protein extracts were prepared as described previously (LaMontagne, et al., Am. J. Pathol., 157, 1937 (2000)).

Statistics: TF was compared with Clark's level, VEGF, P-MAPK, and Breslow thickness using χ² and Fisher's exact tests, and compared with CD31 MVD using a t test. Overall survival and disease-free survival were calculated using the Kaplan-Meier method.

Overall and disease-free survival curves between + and − TFs were compared using log-rank tests, t tests were used to relate CD31 MVD to Clark's level, TF, VEGF, and P-MAPK. One-way ANOVA was used to compare CD31 MVD with Breslow thickness. Cox proportional hazard regression was use to relate CD31 MVD to overall survival and disease-free survival.

Results

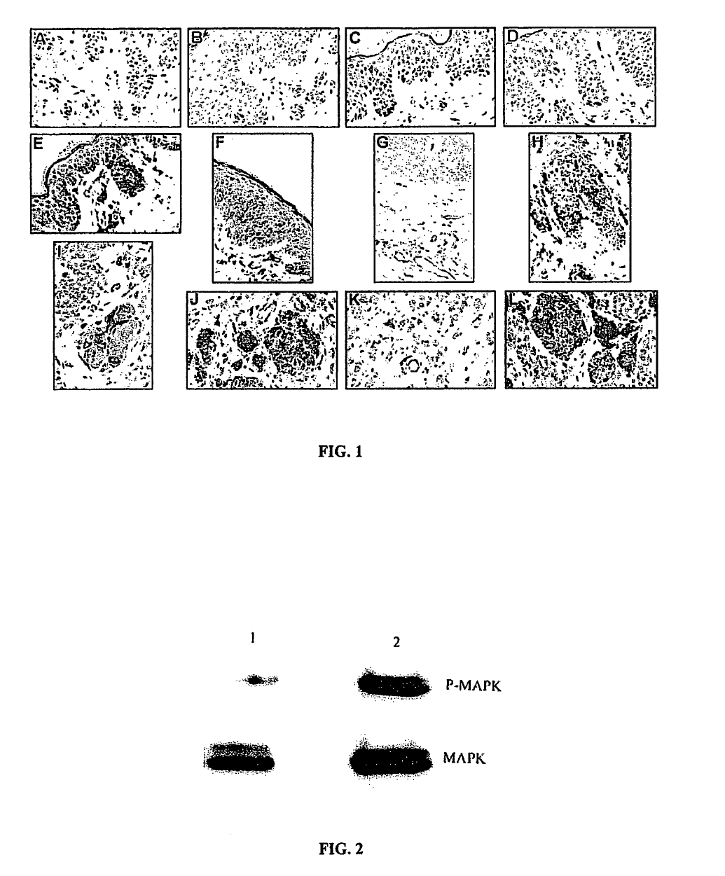

The mean age of the 117 patients studied with MM was 60 years (range, 22-92). Sixty-three (54%) were males, 54 (46%) were females. The Clark level and Breslow depth of invasion of the MM studied are detailed in Table 1, relative to P-MAPK, VEGF, and TF expression. The six atypical nevi were negative for activated MAPK expression, but MAPK activation was noted in both radial and vertical growth phases of MM (FIG. 1) Lymph node status was not available in 32 patients. Follow-up at the time of this report revealed 6 cases of local recurrence and 18 cases of distant metastases among the patients in which follow-up could be obtained. Mean follow-up in 96 patients was 60.8 months (range, 1-227). Expression of P-MAPK was not observed in only 21.5% of benign nevi, all of which had mild atypia, and, thus, were not likely to be diagnostically confused with melanoma (FIG. 1).

Table 2 shows that angiogenesis as CD31 mean MVD correlates significantly with the Clark level of the MM studied (P=0.03) and tended to correlate with TF expression (P −0.06), but showed no significant relationship to Breslow thickness, P-MAPK, and VEGF expression, or overall and disease-free survival (P=>0.05). P-MAPK tended to correlate with Clark level (P=0.08), whereas VEGF did not, but neither VEGF nor P-MAPK expression (angiogenic markers) correlated with Breslow thickness, lymph node status, or overall survival.

Table 3 indicates the statistical relationship between TF and clinical, pathologic, and follow-up parameters. TF expression correlates significantly with Clark level (P=0.019) and VEGF expression (P=0.003), and tended to correlate with angiogenesis as mean MVD of both the mean and maximum CD31 counts (P=0.06), but did not correlate with P-MAPK expression, Breslow thickness, or overall and disease-free survival (P=>0.05). TF expression increased from 28% to 52% to 70% in MM showing VEGF expression of 0-1+, 2+, and 3+ intensity, respectively.

To additionally confirm differences in MAPK signaling between primary melanocytes and radial growth melanoma cells, Western blot analysis were performed by comparing primary human melanocytes with radial growth melanoma (PMWK cells). When cultured in basal medium (DMEM supplemented with 5% FCS), primary melanocytes showed low expression of activated MAPK expression compared with constitutive activation of MAPK in radial growth melanoma cells (FIG. 2)

Discussion of Results

The major cause of death from melanoma is because of distant metastases. The major prognostic markers of melanoma, Breslow thickness and Clark levels, are biological measures of tissue invasion. Melanoma is characterized by a radial growth phase, which proliferates primarily along the dermo-epidermal junction. Radial growth phase melanoma cells accumulate additional mutations, including activation of ras oncogenes and loss of the PTEN tumor suppressor gene. Activation of ras in human and marine melanoma confers the ability of cells to invade the dermis in an expansive and proliferative pattern, and produce angiogenic factors such as VEGF. The ability of melanoma cells to undergo proliferation in three dimensions is clinically known as vertical growth phase melanoma. As expected from experimental data, clinical vertical growth phase melanoma is a highly angiogenic and proliferative lesion.

Several genes have been associated with highly aggressive behavior in vertical growth and metastatic melanoma. These genes include αυβ3 integrin and markers thought previously to be endothelial specific, such as VEGF receptors VEGFR1 and VEGFR2, VE cadherin, and ephrins. This phenomenon has been termed vasculogenic mimicry. Recently, two groups independently isolated rho C through gene chip analysis as a mediator of metastatic behavior. Whereas much knowledge has been gained through these approaches, the events that mark the transition from atypical nevus to early melanoma are not well understood. This is attributable in part to a lack of relevant cell lines, especially because atypical nevi are rarely cultured and do not proliferate well in culture. Recently, Id1, a protein, which down-regulates the tumor suppressor gene p16^{ink4a}, has been shown to be expressed in radial growth melanoma. Down-regulation of p16^{ink4a} may allow MAPK-mediated proliferation and escape from senescence, as activation of MAPK promotes either senescence or transformation, depending on the status of p16^{ink4a}.

It has been discovered that MAPK is a potential mediator of melanocytic tumor progression. Recently, mutations in B-raf have been detected in 59% of melanoma cell lines and 80% of short-term cultures of primary melanomas, and the B-raf mutations is these cells have been shown to cause activation of MAPK signaling. These studies additionally confirm the central role of MAPK signaling in malignant melanoma.

This study has the advantage of determining the timing of MAPK activation in melanoma tumor progression, and has the advantage that these studies can occur in paraffin sections. Targets of MAPK include the proangiogenic markers VEGF and TF. Expression of activated MAPK and its targets, VEGF and TF, are observed in radial growth melanoma and later stages, but not in its immediate precursors. In culture, MAPK activation has been observed in proliferating primary melanocytes in the presence of growth-promoting agents, such as phorbol ester, but is decreased on senescence or removal of growth-promoting agents. In contrast, radial growth melanoma cells grow readily in vitro in the absence of growth-promoting agents. Constitutive expression of activated MAPK is observed in radial growth melanoma cells compared with primary melanocytes.

Decreased expression of activated MAPK has been noted in some specimens in more advanced melanoma. The reasons for this are not currently known, but may include alternative signaling pathways activated in advanced melanoma. Advanced melanomas have been shown previously to express high levels of reactive oxygen species, and it has been shown recently that increased reactive oxygen can stimulate both angiogenesis and tumorigenesis in p16-deficient NIH 3T3 cells. Cells transformed by the reactive oxygen species generating enzyme nox-1 show relatively low levels of MAPK activation, suggesting that reactive oxygen species may assume some of the role of tumorigenesis from MAPK in more advanced lesions.

The findings described here may help explain the conflicting findings between angiogenesis and tumor progression in melanoma. Several studies have implicated a link between prognosis and microvessel density, whereas other studies have not. The findings described here suggest that the angiogenic switch occurs early in melanoma, whereas later events are required for three-dimensional growth and distant metastases. In addition, the findings described here suggest that pharmacologic inhibition of MAPK signaling may be of benefit in the prevention and treatment of cutaneous melanoma.

Many variations and modifications may be made to the above-described embodiments. All such modifications and variations are intended to be included herein within the scope of this disclosure and protected by the following claims.

1. A method of detecting a phosphorylated mitogen activated protein kinase (P-MAPK), comprising:
providing a sample;
contacting the sample with at least one antibody having an affinity for the phosphorylated portion of P-MAPK;
forming an antibody/P-MAPK complex; and
detecting the antibody/P-MAPK complex, wherein the presence of the antibody/P-MAPK complex indicates that the P-MAPK is present in the sample, and wherein constitutive expression of P-MAPK is indicative of cancer.

2. The method of claim 1, wherein the P-MAPK includes variants of P-MAPK, and wherein the at least one antibody has an affinity for the phosphorylated portion of variants of P-MAPK.

3. The method of claim 1, wherein the P-MAPK includes phosphorylated MAPK's selected from MAPK-1 (SEQ ID NO:1), MAPK-3 (SEQ ID NO:2), MAP3K1 (SEQ ID NO:3), MAPK-6 (SEQ ID NO:4), MAPK-8 (SEQ ID NO:5), MAPK-12 (SEQ ID NO:6), MAPK-14 (SEQ ID NO:7), MAPK-14 (SEQ ID NO:8), and combinations thereof.

4. The method of claim 1, wherein the antibody is a detectably labeled antibody.

5. The method of claim 4, wherein the detectably labeled antibody includes a detectable compound selected from a radioisotope label, a metal particle label, a fluorescent label, and an enzyme-substrate label.

6. The method claim 1, wherein the antibody is affixed to a solid support.

7. The method of claim 6, wherein the solid support is selected from a glass, metal, silicon, plastic, latex bead, pins, and dipsticks.

8. The method of claim 1, further comprising:
contacting the sample with at least one antibody having an affinity for a second protein selected from VEGF and TF;
forming an antibody/second protein complex; and
detecting the antibody/second protein complex, wherein the presence of the antibody/P-MAPK complex and the antibody/second protein complex indicates that the P-MAPK is present in the sample, and wherein constitutive expression of P-MAPK and expression of the activated second protein is indicative of cancer.

9. A method of detecting a phosphorylated mitogen activated protein kinase (P-MAPK), comprising:
providing a sample;
contacting the sample with at least one antibody having an affinity for the phosphorylated portion of P-MAPK;
forming an antibody/P-MAPK complex;
contacting the antibody/P-MAPK complex with a detectably labeled antibody having an affinity for the antibody;
forming an antibody/P-MAPK/detectably labeled antibody complex; and
detecting the antibody/P-MAPK/detectably labeled antibody complex, wherein the presence of the antibody/P-MAPK/detectably labeled antibody complex indicates that the P-MAPK is present in the sample, and wherein constitutive expression of P-MAPK is indicative of cancer.

10. The method of claim 9, wherein the P-MAPK includes phosphorylated MAPK's selected from MAPK-1 (SEQ ID NO:1), MAPK-3 (SEQ ID NO:2), MAP3K1 (SEQ ID NO:3), MAPK-6 (SEQ ID NO:4), MAPK-8 (SEQ ID NO:5), MAPK-12 (SEQ ID NO:6), MAPK-14 (SEQ ID NO:7), MAPK-14 (SEQ ID NO:8), and combinations thereof.

11. The method of claim 9, wherein the detectably labeled antibody includes a detectable compound selected from a radioisotope label, a metal particle label, a fluorescent label, and an enzyme-substrate label.

12. The method claim 11, wherein the antibody is affixed to a solid support.

13. The method of claim 12, wherein the solid support is selected from a glass, metal, silicon, plastic, latex bead, pins, and dipsticks.

14. The method of claim 9, further comprising:
contacting the sample with at least one antibody having an affinity for a second protein selected from VEGF and TF;
forming an antibody/second protein complex; and
detecting the antibody/second protein complex, wherein the presence of the antibody/P-MAPK/detectably labeled antibody complex and the antibody/second protein complex indicates that the P-MAPK is present in the sample, and wherein constitutive expression of P-MAPK and expression of the activated second protein is indicative of cancer.

15. A method of diagnosis of cancer, comprising:
providing a sample from a subject; and
determining the presence of a phosphorylated mitogen activated protein kinase (P-MAPK) or a variant thereof in the sample, and wherein constitutive expression of P-MAPK in the sample is indicative of cancer.

16. The method of claim 15, further comprising:
contacting the sample with at least one antibody having an affinity for the phosphorylated portion of P-MAPK or a variant thereof;
forming a complex including the antibody and the P-MAPK or a variant thereof; and
detecting the complex, wherein the presence of the complex indicates the presence of P-MAPK or a variant thereof in the sample.

17. The method of claim 16, further comprising:
contacting the sample with at least one antibody having an affinity for a second protein selected from VEGF and TF;
forming an antibody/second protein complex; and
detecting the antibody/second protein complex, wherein the presence of the complex and the antibody/second protein complex indicates that the P-MAPK is present in the sample, and wherein constitutive expression of P-MAPK and expression of the second protein is indicative of cancer.

18. A kit comprising:
a composition comprising an antibody that bonds to a phosphorylated mitogen activated protein kinase (P-MAPK) or a variant thereof to form a detectable complex; and
a set of printed instructions specifying, in order of implementation, steps to be followed for detecting the P-MAPK or a variant thereof by detecting the complex, wherein the composition and the printed instructions are in packaged combination, and wherein constitutive expression of P-MAPK in a sample is indicative of cancer.

19. The kit of claim 18, further comprising:
a second composition comprising a detectably labeled antibody that bonds with the P-MAPK or a variant thereof to form the detectable complex.

20. The kit claim 18, wherein the antibody is affixed to a solid support.

21. The kit of claim 18, wherein the solid support is selected from a glass, metal, silicon, plastic, latex bead, pins, and dipsticks.

22. The kit of claim 18, further comprising:
a composition comprising an antibody that bonds to a second protein selected from VEGF, TF, and combinations thereof or a variant thereof to form a detectable second protein complex; and
a set of printed instructions specifying, in order of implementation, steps to be followed for detecting the second protein or a variant thereof by detecting the second protein complex, wherein the composition and the printed instructions are in packaged combination, and wherein constitutive expression of P-MAPK and expression of the second protein is indicative of cancer.
